(19) United States
(12) Plant Patent Application Publication
(10) Pub. No.: US 2003/0046744 P1
(43) Pub. Date: Mar. 6, 2003

(54) Petunia plant named 'Kakegawa S28'

(76) Inventor: Masao Bessho, Kakegawa City (JP)

Correspondence Address: JONDLE & ASSOCIATES P.C., 9085 EAST MINERAL CIRCLE, SUITE 200, CENTENNIAL, CO, 80112, US

(21) Appl. No.: 09/946,089

(22) Filed: Sep. 4, 2001

(51) Int. Cl. A01H 5/00

(52) U.S. Cl. PLT/356

(57) ABSTRACT

A Petunia cultivar particularly distinguished by the dark purple flower color.

### BACKGROUND OF THE NEW PLANT

The present invention comprises a new and distinct cultivar of Petunia, botanically known as Petunia hybrida, and hereinafter referred to by the cultivar name ‘Kakegawa S28’. The development of ‘Kakegawa S28’ began with the selection of its male parent. Petunia breeding line Pg-9a-2a was crossed with breeding line 2H-1 in 1994. One F₂ plant from this cross was selected and then crossed to ‘Surfina Violet.’ This hybrid was self pollinated and selected to the F₄ level to produce breeding line 5BC-15A-1A-2. In 1996 this line was hybridized with Kakegawa S28's female parent, a blue-flowered petunia of dwarf habit, known as breeding line 3B-80A-1 b-1 a. These breeding lines are unnamed and unpatented proprietary lines. In 1997 plants from this cross were grown and observed to be segregating for habit and flower characteristics. This breeding work was performed in Kakegawa, Japan.

Two hundred F₁ plants from the 1996 cross were transplanted to a field in Salinas, Calif. during the summer of 1998. Seven lines were selected and vegetatively propagated for further evaluation. The seven lines were propagated again in 1999, and evaluated for fixed characteristics and ease of propagation. Final selection of one line was made during the summer of 1999. The line was established as ‘Kakegawa S28’, and determined to have its characteristics firmly fixed.

‘Kakegawa S28’ has been found to retain its distinctive characteristics after two years and four cycles of vegetative propagation and this novelty is firmly fixed. The variety has demonstrated stability during this time and has no inherent variation or off-types.

### DESCRIPTION OF PHOTOGRAPH

This new Petunia plant is illustrated by the accompanying photograph which shows blooms, and foliage of the plant in full color, the colors shown being as true as can be reasonably obtained by conventional photographic procedures.

FIG. 1 shows the entire plant;

FIG. 2 shows the mature inflorescence.

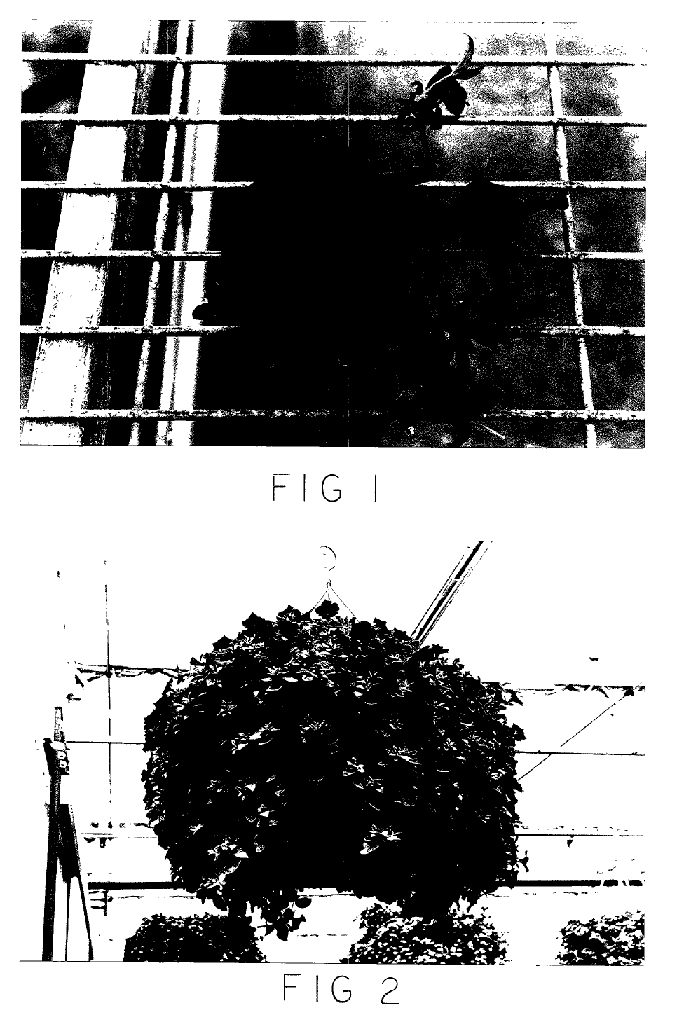

### DESCRIPTION OF THE NEW CULTIVAR

The following detailed descriptions set forth the distinctive characteristics of ‘Kakegawa S28’. The data which defines these characteristics were collected from asexual reproductions carried out in Salinas, Calif. Three plants from fully rooted 15 cm diameter pots were transplanted to one 50 cm diameter hanging baskets and grown in the same conditions. Data was collected on plants in 50 cm diameter pots eight weeks after rooted cuttings were transplanted. Color references are primarily to the RHS Colour Chart of The Royal Horticultural Society of London (RHS).

### Description of the New Plant

Classification: Botanical: Petunia hybrida Commercial: Petunia

Parentage:

Female parent.—breeding line 3B-80A-1 b-1a

Male parent.—breeding line 5BC-15A-1A-2

Environmental Conditions for Plant Growth: Plants were propagated from vegetative cuttings, and grown individually in 15 cm diameter plastic pots in a glass greenhouse located in Salinas, Calif. Pots contained a peat moss-based growing medium. Soluble fertilizer containing 18% nitrogen, 8% phosphorus and 18% potassium was applied in four, daily irrigations. The fifth irrigation was made with non-fertilized water. Pots were top- dressed with a slow release fertilizer containing 18% nitrogen, 8% phosphorus and 18% potassium. The typical average air temperature was 24C.

Growth:

Habit.—branching, mounding

Form.—descending

Plant size.—91 cm total diameter and 76 cm total height when three plants are grown in a 50 cm diameter hanging basket.

Flowering habit.—indeterminate

Time to bloom from propagation.—4-6 weeks after rooting when grown in 10-15 cm diameter plastic pots.

Life cycle.—annual

Stems:

Color.—yellow-green (RHS 145A)

Description.—round, pubescent

Diameter.—2-3 mm

Internode length.—4 cm

Leaves:

Arrangement.—opposite

Apex.—mucronate

Base.—oblique

Color.—upper surface is green (RHS 137B) and lower surface is green (RHS 138B).

Margin.—entire

Size.—length is 4.8 cm and width is 2.5 cm, both at full expansion

Shape.—ovate

Texture.—coarse

Venation.—pinnate

Pubescence.—present, clear

Flowers:

Calyx.—5 sepals; 2 cm×8 mm (length×width)

Corolla.—5 petals, fused

Flower diameter.—6.0 -6.5 cm

Fragrant.—yes

Inflorescence type.—solitary

Pistil.—compound

Ovary.—superior, parietal placentation

Stamens.—5 total with two long and three short

Peduncle.—2.7 cm×2 mm (length33 width); pubescent

Petal color.—Limbs: upper—violet-blue (RHS 89A); lower—violet (RHS 82A) with green (RHS 144C) veins. Tube: Throat—purple (RHS 77A); Inside—purple (RHS 77C) with greyed-purple (RHS 186A) veins; Outside is purple (RHS 77C) with green (RHS 144C) veins.

Petal pubescence.—absent

Tube throat diameter.—1.0 cm

Pollen color.—White (RHS 155C)

Produces seed.—Yes

### Disease and Insect Resistance No susceptibility to diseases or insects noted to date.

Comparison with Known Cultivars ‘Kakegawa S28’ is a distinct variety of Petunia owing to its dark purple flower color and creeping, mounding habit. ‘Kakegawa S28’ is most similar to the variety ‘Purple Wave’; however, ‘Kakegawa S28’ has a darker purple flower color. The table below shows the characteristics that best distinguish the new variety from the comparison variety.

| Characteristic | ‘Kakegawa S28’ | ‘Purple Wave’ |
|---|---|---|
| Flower diameter | 6.0-6.5 cm | 6.2-6.7 cm |
| Primary petal | Dark purple | Deep red-purple RHS 74A |
| color (upper) | RHS 89A | |
| Primary petal | Light purple | Reddish purple RHS 72B |
| color (lower) | RHS 82A | |

I claim:

1. A new and distinct cultivar of Petunia as shown and described herein.
